(19) United States
(12) Patent Application Publication
(10) Pub. No.: US 2015/0100040 A1
(43) Pub. Date: Apr. 9, 2015

(54) MEDICAL MATERIAL SUPPLY DEVICE

(71) Applicant: TERUMO KABUSHIKI KAISHA, Shibuya-ku (JP)

(72) Inventors: Risato KOBAYASHI, Hadano-city (JP); Ichirou HIRAHARA, Setagaya-ku (JP)

(21) Appl. No.: 14/509,550

(22) Filed: Oct. 8, 2014

(30) Foreign Application Priority Data
Oct. 8, 2013 (JP) 2013-211152

(51) Int. Cl. A61M 25/06; A61M 31/00; A61M 25/10; A61M 29/02

(52) U.S. Cl. CPC A61M 25/065; A61M 29/02; A61M 31/002; A61M 25/10; A61M 2029/025; A61M 2025/105; A61M 2025/1086
USPC 604/508; 604/272; 604/57; 604/103.01

(57) ABSTRACT

A medical material supply device which can percutaneously supply a material for nerve regeneration that promotes the regeneration of a nerve is insertable into a vessel and includes: a supply section configured to penetrate from an inner wall to an outer wall of the vessel at a predetermined position in the vessel, thereby being able to supply a material for nerve regeneration to the outer wall of the vessel; and an elongated body which is inserted into the vessel, in which the supply section is provided with a linear portion which is provided at the elongated body and is configured to penetrate from the inner wall to the outer wall of the vessel.

### CROSS-REFERENCES TO RELATED APPLICATIONS

This application claims priority to Japanese Application No. 2013-211152 filed on Oct. 8, 2013, the entire content of which is incorporated herein by reference.

### TECHNICAL FIELD

The present invention relates to a medical material supply device and particularly to a medical material supply device which can supply a material for nerve regeneration that promotes the regeneration of a nerve.

### BACKGROUND DISCUSSION

In order to treat a peripheral nerve that has been cut or that has a defect, it is known to suture the cut nerve or transplant a nerve from other sites.

However, in a case of suturing the cut nerve, it is important to carefully adjust the direction thereof, and thus a high degree of skill is required. Further, in a case of transplanting a nerve collected from another site to a defective portion, it is necessary to perform an operation in two locations, and thus the burden on the patient can be high.

Japanese Patent No. 4721482 discloses a base material in which it is possible to reconstruct a nerve without requiring a high degree of skill and without imposing a heavy burden on a patient. Specifically, Japanese Patent No. 4721482 discloses a base material for nerve reconstruction for reconstructing and joining nerves, in which materials obtained by shaping a material having absorbability in a living body into a fibrous form are bundled.

### SUMMARY

Although surgical treatment involving incision, which regenerates a peripheral nerve as described above, is widely known, percutaneous nerve regeneration treatment without involving incision is not yet established, and thus a burden which is imposed on the body of a patient is still great.

The present disclosure provides for a medical material supply device which can percutaneously supply a material for nerve regeneration that promotes the regeneration of a nerve to the nerve, in view of the above-described problem.

According to an aspect, there is provided a medical material supply device that is inserted into a vessel, including: a supply section configured to penetrate from an inner wall to an outer wall of the vessel at a predetermined position in the vessel, thereby being able to supply a material for nerve regeneration to the outer wall of the vessel; and an elongated body which is inserted into the vessel, in which the supply section is provided with a linear portion which is provided at the elongated body and is configured to penetrate from the inner wall to the outer wall of the vessel.

In an embodiment, the linear portion is provided with a biodegradable core and a layer of a material for nerve regeneration which includes the material for nerve regeneration provided on an outer peripheral surface of the core.

In an embodiment, the linear portion is configured to be separated from the medical material supply device while remaining indwelled after penetrating from the inner wall to the outer wall of the vessel at the predetermined position.

In an embodiment, the elongated body defines a hollow portion inside thereof and the linear portion defines a hollow portion which communicates with the hollow portion of the elongated body and also communicates with a distal end of the linear portion, and thus the material for nerve regeneration can be supplied from the hollow portion of the elongated body to the outer wall of the vessel through the hollow portion of the linear portion.

In an embodiment, the linear portion comprises a distal end portion which includes a distal end of the elongated body, and has a spiral shape.

In an embodiment, the linear portion extends at a predetermined angle with respect to an extension direction of the elongated body.

In an embodiment, the supply section is provided with a dilation portion which retains the linear portion and dilates at the predetermined position in the vessel and the dilation portion can dilate, whereby the distal end of the linear portion penetrates from the inner wall to the outer wall of the vessel.

In an embodiment, the elongated body defines a hollow portion inside thereof and the dilation portion is provided with a balloon which is provided at an outer wall of the elongated body and defines a dilated space to which a liquid is supplied from the hollow portion through a hole connecting the outer wall and an inner wall of the elongated body.

If a medical material supply device according to an embodiment is used, it is possible to perform a method of supplying a material for nerve regeneration to a nerve which includes a step of inserting the medical material supply device into a vessel, thereby introducing the medical material supply device at a predetermined position in the vessel, and a step of making a tip of a linear portion provided in the medical material supply device penetrate from an inner wall to an outer wall of the vessel at the predetermined position, thereby supplying the material for nerve regeneration from the linear portion to the outer wall of the vessel. Further, in an embodiment, the method further includes a step of stopping the neural activity of a nerve extending along the outer wall of the vessel before the supply of the material for nerve regeneration to the outer wall of the vessel.

According to such a medical material supply device, it becomes possible to percutaneously supply a material for nerve regeneration which promotes the regeneration of a nerve.

An embodiment of a medical treatment method using a medical material supply device includes: inserting the medical material supply device into a vessel; penetrating from an inner wall to an outer wall of the vessel with a linear portion of the medical material supply device; and supplying medical material to the vessel with the linear portion.

An embodiment of a medical treatment method further includes, after penetrating from the inner wall to the outer wall of the vessel with the linear portion of the medical material supply device, winding the linear portion around the vessel.

An embodiment of a medical treatment method further includes, after penetrating from the inner wall to the outer wall of the vessel with the linear portion of the medical material supply device, separating the linear portion from the medical material supply device.

An embodiment of a medical treatment method further includes penetrating from the inner wall to the outer wall of the vessel with a plurality of linear portions.

In an embodiment of a medical treatment method, the linear portion has a hollow portion and the medical material is supplied to the vessel by being supplied to the hollow portion of the linear portion.

In an embodiment of a medical treatment method, the linear portion is provided with an outer layer of the medical material which is supplied to the vessel.

### BRIEF DESCRIPTION OF DRAWINGS

FIGS. 1(a) and 1(b) are diagrams illustrating a medical material supply device 1 as a first embodiment.

FIGS. 2(a) and 2(b) are diagrams illustrating a medical material supply device 11 as a second embodiment.

FIGS. 3(a) and 3(b) are diagrams illustrating a medical material supply device 21 as a third embodiment.

FIGS. 4(a) and 4(b) are diagrams illustrating a medical material supply device 31 as a fourth embodiment.

FIG. 5 is a cross-sectional view illustrating an example of a linear portion 15 of the medical material supply device 11.

FIG. 6 is a cross-sectional view illustrating another example of the linear portion 15.

FIG. 7 is a diagram illustrating an example of the disposition of a linear portion 25 in the medical material supply device 21.

FIG. 8 is a diagram illustrating a balloon 54 of the medical material supply device 21 which is in a state of being folded in a blood vessel BV.

FIG. 9 is a diagram illustrating an example of a linear portion which can be used in the medical material supply device 21.

FIG. 10 is a diagram illustrating an example of a dilation portion which can be used in the medical material supply device 21.

FIG. 11 is a diagram illustrating treatment to supply a material for nerve regeneration to a renal artery sympathetic nerve NE which is in an inactivation state, by using the medical material supply device 1.

FIG. 12 is a diagram illustrating treatment to perform the denervation of the renal artery sympathetic nerve NE.

### DETAILED DESCRIPTION

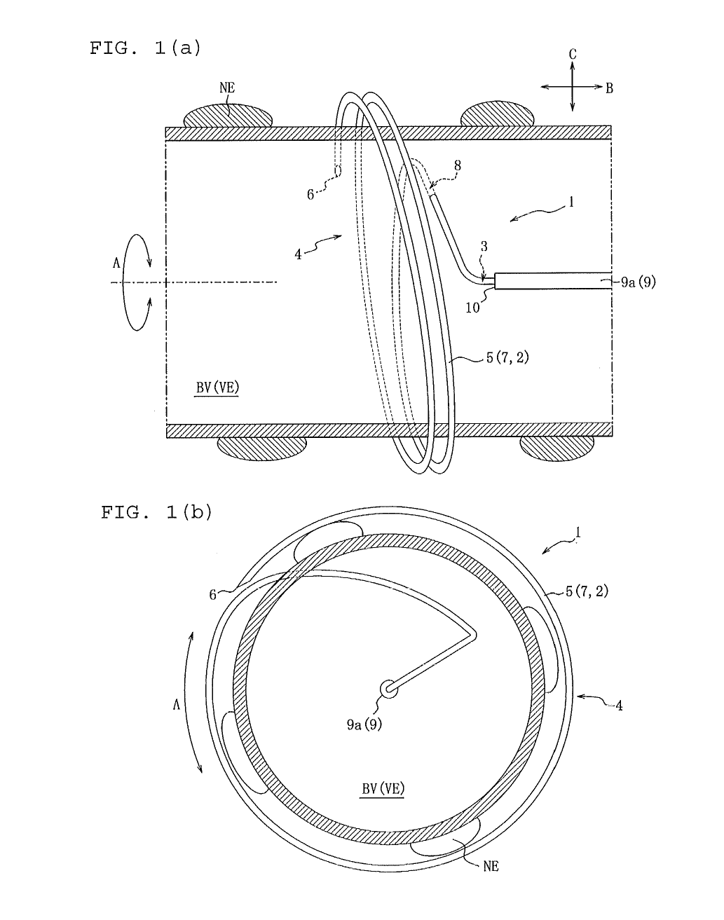

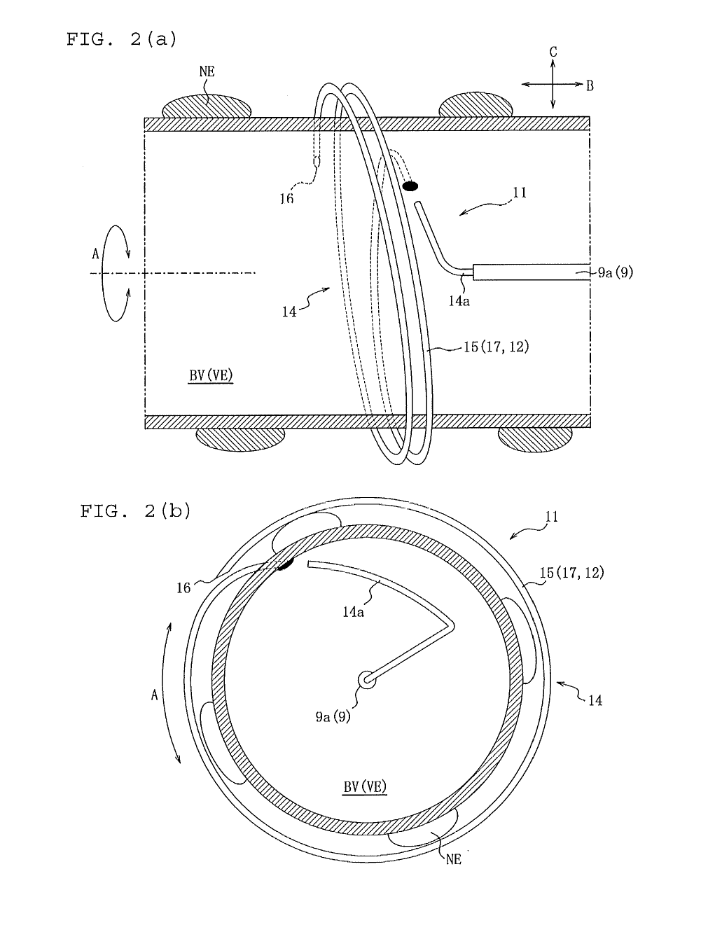

Hereinafter, embodiments of a medical material supply device according to the present disclosure will be described with reference to FIGS. 1 to 12. In addition, in each drawing, a common member or site will be denoted by the same number or symbol.

FIGS. 1 to 4 respectively show first to fourth embodiments of a medical material supply device. All medical material supply devices 1, 11, 21, and 31 in the first to fourth embodiments are medical material supply devices which are inserted into a vessel VE, and are provided with supply sections 2, 12, 22, and 32, each of which penetrates from an inner wall to an outer wall of the vessel VE at a predetermined position in the vessel VE, thereby being able to supply a material for nerve regeneration to the outer wall of the vessel VE.

In this manner, all the medical material supply devices 1, 11, 21, and 31 shown in the respective first to fourth embodiments are percutaneously introduced into the vessel VE, penetrate a vessel wall at a predetermined position, thereby protruding from the inside of the vessel VE to the outside of the vessel VE, and can supply the material for nerve regeneration to a nerve which is located in the vicinity of the outer wall of the vessel VE.

Here, the “vessel” refers to a tube which is present in the body and permits the passage of a body fluid, and is, for example, a blood vessel, a lymphatic vessel, or the like. Further, the “material for nerve regeneration” refers to a material which promotes the regeneration of a nerve, and, for example is a neural inducer, a scaffold material, or the like. More specifically, a material such as laminin, fibronectin, alginic acid, or poly-L-lysine can be given as an example thereof. In addition, the “scaffold material” refers to a material for becoming a scaffold which promotes differentiation and induction of a cell, thereby regenerating a tissue, in regenerative medicine, and a living body-derived extracellular matrix or a synthetic chemical material with high bio-absorbability can be given as an example thereof.

Hereinafter, the respective first to fourth embodiments will be described in detail with reference to FIGS. 1 to 10.

FIG. 1 is a diagram illustrating the medical material supply device 1 as the first embodiment. Specifically, FIG. 1(a) is a diagram illustrating the medical material supply device 1 inserted into a blood vessel BV as the vessel VE, and FIG. 1(b) is a schematic diagram illustrating a positional relationship in a circumferential direction A of the blood vessel BV between the medical material supply device 1 which is in the same state as in FIG. 1(a) and the blood vessel BV.

As shown in FIG. 1(a), the medical material supply device 1 is provided with an elongated body 4 which is inserted into the blood vessel BV from the outside of the body and defines a hollow portion 3 inside thereof, and the supply section 2 is provided at the elongated body 4. Specifically, the supply section 2 is provided with a linear portion 5 which penetrates from an inner wall to an outer wall of the blood vessel BV. In this embodiment, the supply section 2 itself is the linear portion 5 which comprises a distal end portion 7 that includes a distal end of the elongated body 4, and which has a spiral shape. In addition, the elongated body 4 is a tubular member such as a catheter, for example.

The linear portion 5 defines a hollow portion 8 which communicates with the hollow portion 3 of the elongated body 4 and also communicates with a distal end 6 of the linear portion 5 (in this embodiment, the same as the distal end of the elongated body 4).

As shown in FIGS. 1(a) and 1(b), the distal end 6 of the linear portion 5 (the supply section 2) in the medical material supply device 1 is located in the vicinity of the outer wall of the blood vessel BV and penetrates from the inner wall to the outer wall of the blood vessel BV at a predetermined position in the blood vessel BV in the vicinity of a nerve NE so as to be able to supply the material for nerve regeneration to the nerve NE in which neural activity is stopped or which is in an inactivation state. Specifically, if the distal end 6 of the linear portion 5 having a spiral shape is brought into contact with the inner wall of the blood vessel BV at a predetermined position and axial rotation of the elongated body 4 is performed from the state, the linear portion 5 also rotates due to the rotational motion, and thus the distal end 6 of the linear portion 5 penetrates from the inner wall to the outer wall of the blood vessel BV. If the elongated body 4 is further rotated, the distal end 6 of the linear portion 5 advances to one side (in FIG. 1, the left side) in an extension (i.e., longitudinal) direction B of the blood vessel BV while being spirally wound along the outer peripheral surface of the blood vessel BV. In addition, in a case of retreating the linear portion 5 to the other side (in FIG. 1, the right side), it has only to perform the axial rotation of the elongated body 4 in the opposite direction to the direction when advancing the distal end 6 of the linear portion 5.

As described above, since the linear portion 5 defines the hollow portion 8 which communicates with the hollow portion 3 of the elongated body 4 and also communicates with the distal end 6 of the linear portion 5 (in this embodiment, the same as the distal end of the elongated body 4), the material for nerve regeneration which is supplied from the proximal end side of the elongated body 4 through the hollow portion 3 is transported from the inside of the blood vessel BV to the outside of the blood vessel BV through the hollow portion 8 of the linear portion 5 of a spiral shape wound along the outer peripheral surface of the bold vessel BV from the hollow portion 3 of the elongated body 4, and is supplied from an opening of the hollow portion 8 which is located at the distal end 6 of the linear portion 5 to the outer wall of the blood vessel BV. In this way, it becomes possible to percutaneously supply the material for nerve regeneration to the nerve NE which is located in the vicinity of the outer wall of the blood vessel BV and in which neural activity is stopped or which is in an inactivation state.

Specifically, the material for nerve regeneration is supplied onto the outer wall of the blood vessel BV by retreating the linear portion 5 while sending the material for nerve regeneration in the form of a gel or the form of a liquid from the distal end 6 of the linear portion 5 wound along the outer peripheral surface of the blood vessel BV. The distal end 6 of the linear portion 5 is pulled back into the blood vessel BV as it is after the supply of the material for nerve regeneration and is removed to the outside of the body through the blood vessel BV.

Here, the linear portion 5 having a spiral shape advances to one side in the extension direction B of the blood vessel BV so as to cover at least the entire area in the circumferential direction A of the blood vessel BV, as shown in FIG. 1(b). By doing so, it is possible to supply the material for nerve regeneration to the entire area in the circumferential direction A of the blood vessel BV. Therefore, it is possible to reliably supply the material for nerve regeneration to the nerve NE regardless of the position in the circumferential direction A of the blood vessel BV, of the nerve NE which extends substantially parallel to the extension direction B of the blood vessel BV and in which neural activity is stopped or which is in an inactivation state.

Further, as shown in FIG. 1(b), the distal end 6 of the linear portion 5 in this embodiment is configured so as to be located further to the outer peripheral surface side of the linear portion 5 having a spiral shape than a central axis I in the extension (i.e., longitudinal) direction B of the linear portion 5. With this configuration, it becomes easy for the distal end 6 of the linear portion 5 to come into contact with the inner wall of the blood vessel BV when penetrating from the inner wall of the blood vessel BV to the outer wall of the blood vessel BV, and thus it is easy to make the distal end 6 of the linear portion 5 penetrate from the inner wall to the outer wall of the blood vessel BV by the axial rotation motion of the elongated body 4 at a predetermined position in the blood vessel BV. In addition, when advancing the distal end 6 of the linear portion 5 to one side in the extension direction B of the blood vessel BV after the penetration of the distal end 6 of the linear portion 5, it becomes difficult for the distal end 6 of the linear portion 5 to penetrate from the outer wall of the blood vessel BV to the inner wall of the blood vessel BV again, and thus the distal end 6 of the linear portion 5 is prevented from puncturing the outer wall of a blood vessel wall by mistake.

In addition, in this embodiment, the medical material supply device 1 is provided with a transport member 9 which transports the linear portion 5 (the supply section 2) to a predetermined position in the blood vessel BV in an elastically deformed state. The linear portion 5 (the supply section 2) in this embodiment includes the distal end portion 7 of the elongated body 4 and is inserted from the outside of the body at a predetermined position in the blood vessel BV through a hollow portion of a guiding catheter 9a as the transport member 9. The linear portion 5 is shaped so as to maintain a spiral shape as shown in FIG. 1(a) in a state where an external force does not act thereon at all. The inner diameter of a substantially cylindrical hollow portion which is defined by the linear portion 5 having a spiral shape is configured so as to be substantially equal to the outer diameter of the blood vessel BV. Further, the linear portion 5 has flexibility in which it can be elastically deformed if an external force acts thereon. Therefore, in the guiding catheter 9a, the linear portion 5 is elastically deformed by being pressed by an inner wall of the guiding catheter 9a, thereby having a line shape along the guiding catheter 9a. However, if the distal end 6 of the linear portion 5 passes through a distal end 10 of the guiding catheter 9a, regulation by the inner wall of the guiding catheter 9a is released, and thus the shape thereof changes into a spiral shape due to a restoring force. Then, the linear portion 5 turned into a spiral shape enters a state of pressing the inner wall of the blood vessel BV to the outside in a radial direction C of the blood vessel BV at a predetermined position in the blood vessel BV, and by performing the axial rotation of the elongated body 4 from this state, it is possible to make the distal end 6 of the linear portion 5 penetrate from the inner wall to the outer wall of the blood vessel BV.

On the contrary, when removing the elongated body 4 which includes the linear portion 5 to the outside of the body, after the linear portion 5 is pulled back into the blood vessel BV, if the linear portion 5 is drawn out in a state where the guiding catheter 9a is indwelled, the linear portion 5 is elastically deformed so as to be accommodated in the hollow portion of the guiding catheter 9a again, thereby being able to be removed to the outside of the body.

In addition, as the transport member 9, in addition to using the guiding catheter 9a described above, a configuration is also acceptable in which an outer cylindrical member which is inserted at a predetermined position of the blood vessel BV with the linear portion 5 (the supply section 2) accommodated inside is separately provided. By inserting the outer cylindrical member which accommodates the linear portion 5 inside in a state of being elastically deformed in a substantially straight line shape, at a predetermined position in the blood vessel BV through the guiding catheter 9a, and then removing only the outer cylindrical member to the outside of the body through the guiding catheter 9a with the linear portion 5 left behind at the predetermined position, it is possible to change the shape of the linear portion 5 into a spiral shape at a predetermined position in the blood vessel BV. When recovering the elongated body 4 which includes the linear portion 5, the outer cylindrical member is inserted again at a predetermined position in the blood vessel BV through the guiding catheter 9a. Subsequently, by elastically deforming the linear portion 5 having a spiral shape in a substantially straight line shape, thereby accommodating the linear portion 5 in the outer cylindrical member again, and removing the outer cylindrical member to the outside of the body through the guiding catheter 9a in a state where the outer cylindrical member accommodates the linear portion 5 inside, the recovery of the elongated body 4 which includes the linear portion 5 is completed.

Here, a material of the supply section 2 (the linear portion 5) in this embodiment can be assumed to principally include at least one kind of material which is selected from the group consisting of a polyolefin such as high density polyethylene or polypropylene; a polyamide such as nylon 66; polyurethane; a polyester such as polyethylene terephthalate, polybutylene terephthalate, or polycyclohexane terephthalate; and a fluorine-based resin such as polytetrafluoroethylene or ethylene-tetrafluoroethylene copolymer. In addition, the inner surface and/or the outer surface of the supply section 2 (the linear portion 5) may be coated with an antithrombotic resin film so as to secure biocompatibility.

Further, as the material of the supply section 2 (the linear portion 5), in addition to the resin materials described above, for example, stainless steel, tantalum titanium, a nickel titanium alloy, elastic metal, or the like can be used, and one kind of these may be used alone and two kinds or more may also be used in combination. Among these, elastic metal is preferable and a super-elastic alloy is more preferable. The super-elastic alloy is generally called a shape memory alloy and exhibits elasticity at least a living body temperature (around 37° C.). As the super-elastic alloy, although there is no particular limitation, a titanium-nickel alloy containing 49 atomic % to 53 atomic % of nickel is preferable.

As the buckling strength (yield stress under load) of the super-elastic alloy, there is no particular limitation and it can be appropriately selected according to the purpose. However, a range of 3 kg/mm2 to 20 kg/mm2 (22° C.) is preferable. As the restoring stress (yield stress under no load) of the super-elastic alloy, there is no particular limitation and it can be appropriately selected according to the purpose. However, a range of 3 kg/mm2 to 180 kg/mm2 (22° C.) is preferable. In addition, “super-elasticity” means that, even if at an operating temperature, normal metal is deformed (bent, pulled, or compressed) to a domain in which it is plastically deformed, after the release of the deformation, an almost original shape is recovered without needing heating.

In addition, in this embodiment, since the supply section 2 (the linear portion 5) comprises the distal end portion 7 of the elongated body 4, the above-described material of the supply section 2 (the linear portion 5) can also be used as a material of the elongated body 4.

Further, the guiding catheter 9a as the transport member 9 has flexibility and can be formed of, for example, a polyolefin such as polyethylene, polypropylene, polybutene, ethylene-propylene copolymer, ethylene-vinyl acetate copolymer, an ionomer, or a mixture of two kinds or more of these, a thermoplastic resin such as soft polyvinyl chloride resin, polyamide, a polyamide elastomer, polyester, a polyester elastomer, polyurethane, a fluorine resin, or an acrylic resin, silicone rubber, latex rubber, or the like.

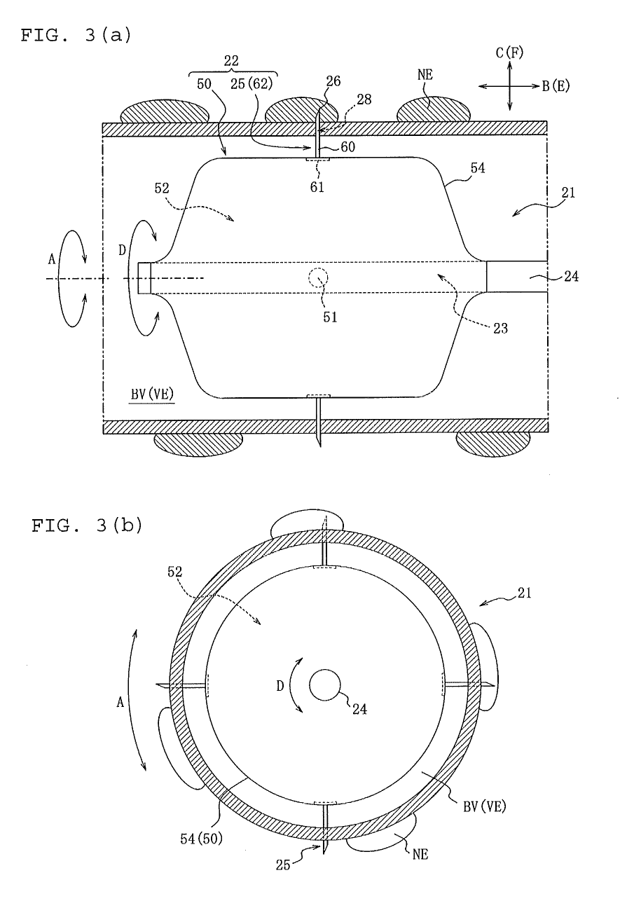

Next, the medical material supply device 11 as the second embodiment will be described with reference to FIG. 2. The medical material supply device 11 in this embodiment is different in the configuration of the supply section from the medical material supply device 1 in the first embodiment. Here, the difference will be mainly described, and with respect to a configuration shared by the medical material supply device 1 as the first embodiment, description thereof will be omitted.

FIG. 2 is a diagram illustrating the medical material supply device 11 as the second embodiment. Specifically, FIG. 2(a) is a diagram illustrating the medical material supply device 11 inserted into the blood vessel BV as the vessel VE, and FIG. 2(b) is a schematic diagram illustrating a positional relationship in the circumferential direction A of the blood vessel BV between the medical material supply device 11 which is in the same state as in FIG. 2(a) and the blood vessel BV.

As shown in FIG. 2(a), the medical material supply device 11 is provided with an elongated body 14 which is inserted into the blood vessel BV from the outside of the body, and a supply section 12 is provided at the elongated body 14. Specifically, the supply section 12 is provided with a linear portion 15 which penetrates from the inner wall to the outer wall of the blood vessel BV. In this embodiment, the supply section 12 itself is the linear portion 15 which comprises a distal end portion 17 that includes a distal end of the elongated body 14, and which has a spiral shape. In addition, as the elongated body 14, similar to the first embodiment, for example, a tubular member such as a catheter can be used. However, in this embodiment, a guide wire having no hollow portion is used.

FIG. 5 is a diagram illustrating the cross-section of the linear portion 15. As shown in FIGS. 2(a) and 2(b), the shape of the linear portion 15 (the supply section 12) has the same spiral shape as in the linear portion 5 (the supply section 2) in the first embodiment. However, as shown in FIG. 5, the linear portion 15 is different from the linear portion 5 in the first embodiment in that the linear portion 15 is a solid linear portion having no hollow portion and that the linear portion 15 is configured to include a biodegradable core 15a and a layer of a material for nerve regeneration 15b which includes a material for nerve regeneration and which is provided on the outer peripheral surface of the core 15a. In addition, a linear portion 35 in the fourth embodiment which will be described later also has the same configuration as the cross-section of the linear portion 15 shown in FIG. 5.

In the linear portion 15 in this embodiment, similar to the linear portion 5 in the first embodiment, a distal end 16 of the linear portion 15 penetrates from the inner wall to the outer wall of the blood vessel BV due to performing axial rotation of the elongated body 14 at a predetermined position in the blood vessel BV, and the distal end 16 of the linear portion 15 advances to one side (the left side in FIG. 2(a)) in the extension direction B of the blood vessel BV by further performing the axial rotation of the elongated body 14 in the same direction.

In this embodiment, after the linear portion 15 advances, thereby being wound along the outer peripheral surface of the blood vessel BV, the linear portion 15 is separated from the medical material supply device 11 while indwelled at the above-described predetermined position, and the medical material supply device 11 with the linear portion 15 separated therefrom (in this embodiment, only an elongated body main body 14a with the linear portion 15 separated therefrom) is removed and recovered from the blood vessel BV.

As described above, the linear portion 15 is provided with the biodegradable core 15a and the layer of a material for nerve regeneration 15b which includes the material for nerve regeneration and which is provided on the outer peripheral surface of the core 15a and the linear portion 15 is indwelled in a state of being wound around the outer peripheral surface of the blood vessel BV, and therefore, the layer of a material for nerve regeneration 15b in the form of a gel or the form of a solid diffuses into the body, and as a result, the material for nerve regeneration is supplied to the nerve NE which is located in the vicinity of the outer wall of the blood vessel BV, thereby promoting the regeneration of the nerve NE in which neural activity is stopped or which is in an inactivation state.

The core 15a is configured with, for example, a biodegradable polymer as a biodegradable material. The biodegradable polymer means a polymer which is gradually degraded when the linear portion 15 is indwelled by being wound around the outer peripheral surface of the blood vessel BV and which is a polymer that does not adversely affect the living body of a human being or an animal.

The biodegradable polymer which is used for the core 15a is not particularly limited. However, at least one which is selected from the group consisting of polyglycolic acid, polylactic acid, polycaprolactone, polyhydroxy butyric acid, cellulose, polyhydroxybutyrate valerate, and a polyorthoester, or a copolymer, a mixture, or a combination of these is preferable. Further, it is preferable that these biodegradable polymers have a tensile strength greater than or equal to a certain level. For example, in a case of polylactic acid, it is preferable that the tensile strength be greater than or equal to 55 Mpa.

Further, the biodegradable polymer may contain a plasticizer. If it contains a plasticizer, the ductility of the biodegradable polymer is improved, and thus it is possible to improve the bending flexibility of the linear portion 15. As the plasticizer, as long as it is a plasticizer which does not adversely affect the living body of a human being or an animal, there is no particular limitation. However, at least one which is selected from the group consisting of polyethylene glycol, polyoxyethylene polyoxypropylene glycol, polyoxyethylene sorbitan monooleate, polyethylene glyceryl triricinolate, sorbitan sesquioleate, triethyl citrate, acetyl tributyl citrate, acetyl trihexyl citrate, butyryl trihexyl citrate, a medium-chain triglyceride, a monoglyceride, and an acetylated monoglyceride, or a mixture of these is preferable. Such a plasticizer is used so as to be contained in a range of 0.01% by mass to 80% by mass, preferably, 0.1% by mass to 60% by mass, and further preferably, 1% by mass to 40% by mass with respect to a biodegradable polymer material.

The layer of a material for nerve regeneration 15b can be formed, for example, by laminating a layer obtained by making a material for nerve regeneration be contained in a biodegradable polymer on the outer surface of the core 15a by, for example, a coating process. Further, the layer of a material for nerve regeneration 15b can also be laminated on the outer surface of the core 15a as a layer in the form of a gel or the form of a solid, which is made of only a material for nerve regeneration. In addition, the layer of a material for nerve regeneration 15b in which plural kinds of material for nerve regeneration are mixed is also acceptable.

Here, the linear portion 15 is bonded to the elongated body 14a by an adhesive which is softened by heat. Therefore, in the linear portion 15, after the distal end 16 of the linear portion 15 penetrates from the inner wall to the outer wall of the blood vessel BV at a predetermined position in the blood vessel BV, specifically, after the distal end 16 of the linear portion 15 advances to one side in the extension direction B of the blood vessel BV, whereby the linear portion 15 is wound along the outer peripheral surface of the blood vessel BV until the linear portion 15 having a spiral shape covers the entire area in the circumferential direction A of the blood vessel BV, as shown in FIG. 2(b), heat is generated (for example, in a range of 45° C. to 50° C.) between the linear portion 15 and the elongated body main body 14a in the elongated body 14 by making an electric current flow through the elongated body 14, whereby the adhesive is softened, and thus the linear portion 15 is separated from the medical material supply device 11. With this configuration, it becomes possible to easily separate the linear portion 15 from the medical material supply device 11.

In addition, in this embodiment, the linear portion 15 can be separated from the medical material supply device 11 by an electric current. However, for example, a configuration is also acceptable in which the linear portion 15 and the elongated body main body 14a are bonded to each other by an adhesive which is decomposed if it touches a liquid such as blood. In the case of such a configuration, a bonded portion is covered by the guiding catheter 9a until the timing to separate the linear portion 15 from the medical material supply device 11 such that blood in the blood vessel BV first touches the bonded portion when separating the linear portion 15 from the medical material supply device 11.

Further, a configuration is also acceptable in which, for example, a thinnest portion which is the thinnest in the elongated body 14 is provided between the linear portion 15 and the elongated body main body 14a so as to be dynamically easily cut thereat and the thinnest portion is easily cut by pulling the elongated body main body 14a after the linear portion 15 is wound along the outer peripheral surface of the blood vessel BV. Furthermore, a configuration is also acceptable in which a separation mechanism capable of separating the linear portion 15 from the medical material supply device 11 at a predetermined position in the blood vessel BV is provided between the linear portion 15 and the elongated body main body 14a.

In addition, it is preferable to provide a coming-out prevention portion is proximal end portion of the linear portion 15 to be separated from the medical material supply device 11 while remaining indwelled, that is, a portion separated from the elongated body 14 so as not to come out from the inside of the blood vessel BV to the outside of the blood vessel BV.

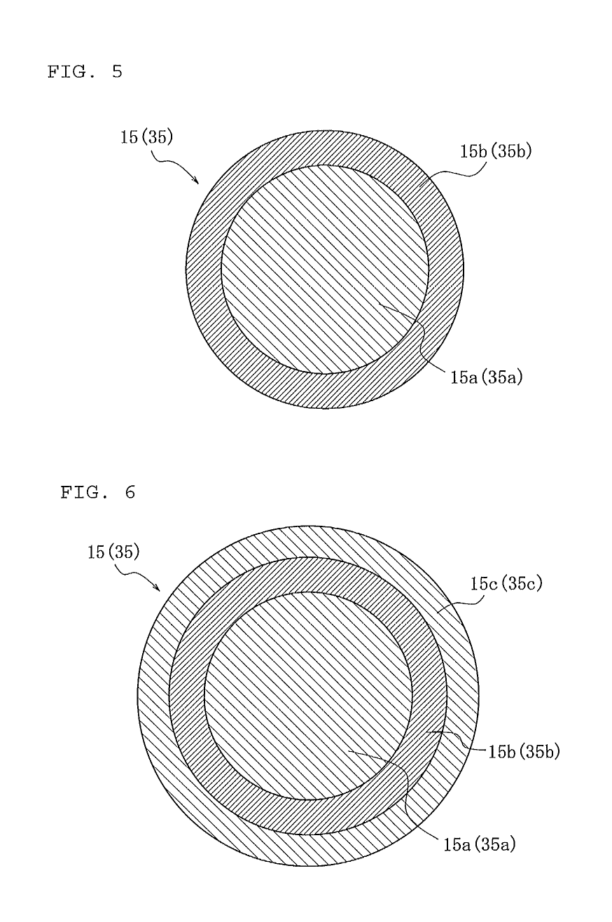

Further, as shown in FIG. 6, the linear portion 15 of this embodiment has a two-layer structure having the core 15a and the layer of a material for nerve regeneration 15b, but, may have, for example, a configuration in which a neurotoxin layer 15c is laminated outside the layer of a material for nerve regeneration 15b.

Recently, treatment to inactivate a renal artery sympathetic nerve has been performed on a resistant hypertensive patient. However, a fall in blood pressure appears over a period from several weeks to several months after treatment. The inventor has performed extensive studies focusing on the fact that there is no effect of a fall in blood pressure immediately even if a nerve is inactivated, and as a result, has obtained the knowledge that the regeneration of an inactivated nerve is related to a fall in blood pressure. This is because it is found that a nerve inactivated by treatment is regenerated in a period from two weeks to three months and a function of the regenerated nerve is different from a function of a nerve before being inactivated. In addition, inactivating a nerve is hereinafter referred to as “denervation”.

Therefore, if the neurotoxin layer 15c is provided outside the layer of a material for nerve regeneration 15b of the linear portion 15, as described above, first, it is possible to create an inactivation state by stopping the neural activity of the nerve NE which is located in the vicinity of the outer wall of the blood vessel BV by the action of the neurotoxin layer 15c decomposed by heat or the like. Thereafter, the regeneration of the nerve NE which is in an inactivation state is promoted by the action of the layer of a material for nerve regeneration 15b described above. In addition, also with respect to the linear portion 35 in the fourth embodiment which will be described later, it is possible to adopt the same configuration as that of the linear portion 15 shown in FIG. 6.

The neurotoxin layer 15c can be formed, for example, by laminating a layer obtained by making ethanol as a neurotoxin be contained in a biodegradable polymer on the outer surface of the core 15a by, for example, a coating process. Further, in a case of using phenol, guanethidine, or the like as the neurotoxin, the neurotoxin layer 15c in the form of a gel or the form of a solid, which is configured with one material of these materials, may be laminated on the outer surface of the layer of a material for nerve regeneration 15b. In addition, the neurotoxin 15c in which plural kinds of neurotoxic materials such as phenol or guanethidine are mixed is also acceptable. Furthermore, the neurotoxin layer 15c formed by making a neurotoxic material other than ethanol be contained in a biodegradable polymer in the same way as a layer formed by making ethanol be contained in the biodegradable polymer described above is also acceptable.

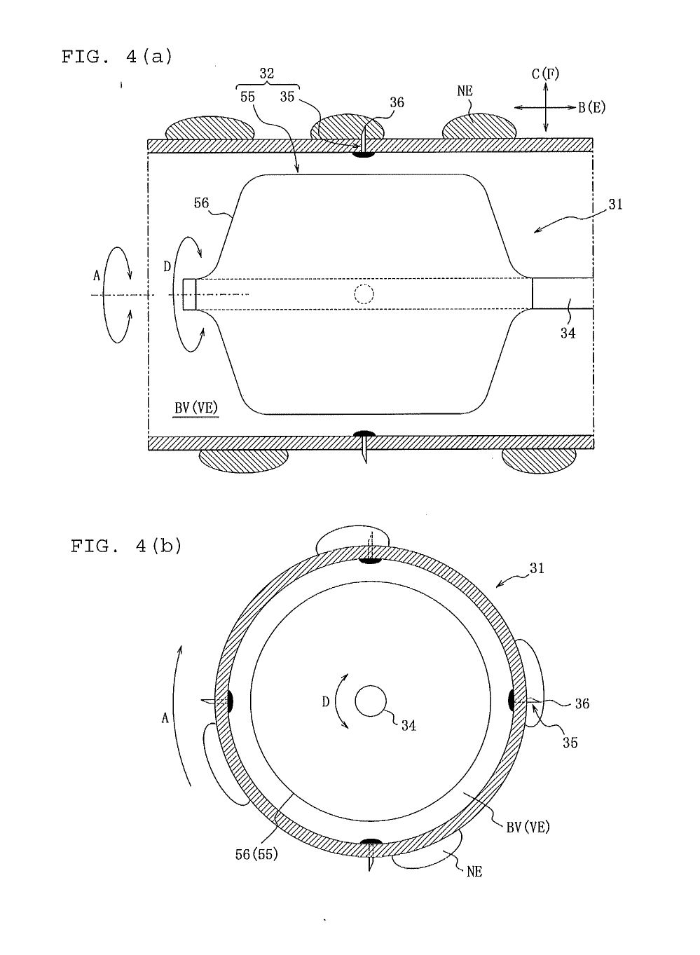

Next, the medical material supply device 21 as the third embodiment will be described with reference to FIG. 3. FIG. 3 is a diagram illustrating the medical material supply device 21 as the third embodiment. Specifically, FIG. 3(a) is a diagram illustrating the medical material supply device 21 inserted into the blood vessel BV as the vessel VE, and FIG. 3(b) is a schematic diagram illustrating a positional relationship in the circumferential direction A of the blood vessel BV between the medical material supply device 21 which is in the same state as in FIG. 3(a) and the blood vessel BV.

As shown in FIG. 3(a), the medical material supply device 21 is provided with an elongated body 24 which is inserted into the blood vessel BV from the outside of the body and defines a hollow portion 23 inside thereof, and a supply section 22 is provided at an outer wall of the elongated body 24. In addition, the elongated body 24 is a tubular member such as a catheter, for example.

The supply section 22 is provided with a linear portion 25 which penetrates from the inner wall to the outer wall of the blood vessel BV, and a dilation portion 50 which retains the linear portion 25 and dilates at a predetermined position in the blood vessel BV.

The linear portion 25 defines a hollow portion 28 which communicates with the hollow portion 23 of the elongated body 24 and also communicates with a distal end 26 of the linear portion 25.

The dilation portion 50 is provided with a balloon 54 which is provided at an outer wall of the elongated body 24 and defines a dilated space 52 to which a liquid is supplied from the hollow portion 23 of the elongated body 24 through a hole 51 which connects the outer wall and an inner wall of the elongated body 24.

As shown in FIGS. 3(a) and 3(b), a liquid of the material for nerve regeneration is supplied to the dilated space 52 that the balloon 54 defines, at a predetermined position in the blood vessel BV in the vicinity of the nerve NE which is located in the vicinity of the outer wall of the blood vessel BV and in which neural activity is stopped or which is in an inactivation state, such that the distal end 26 of the linear portion 25 in the supply section 22 of the medical material supply device 21 can supply the material for nerve regeneration to the nerve NE, and thus the dilation portion 50 dilates, whereby the distal end 26 of the linear portion 25 presses the inner wall of the blood vessel BV, and as a result, the distal end 26 of the linear portion 25 becomes stuck in the inner wall of the blood vessel BV, and thus the distal end 26 of the linear portion 25 penetrates from the inner wall to the outer wall of the blood vessel BV.

As described above, since the linear portion 25 defines the hollow portion 28 which communicates with the hollow portion 23 of the elongated body 24 and also communicates with the distal end 26 of the linear portion 25, the material for nerve regeneration which is supplied from the proximal end side of the elongated body 24 through the hollow portion 23 is transported from the inside of the blood vessel BV to the outside of the blood vessel BV by passing through the hollow portion 28 of the linear portion 25 from the hollow portion 23 of the elongated body 24 in a state where the distal end 26 of the linear portion 25 penetrates a blood vessel wall of the blood vessel BV. Thereafter, the material for nerve regeneration comes out from an opening of the hollow portion 28 which is located at the distal end 26 of the linear portion 25, and is supplied to the outer wall of the blood vessel BV. In this way, it becomes possible to percutaneously supply the material for nerve regeneration to the nerve NE which is located in the vicinity of the outer wall of the blood vessel BV and in which neural activity is stopped or which is in an inactivation state. In particular, in this embodiment, since the hollow portion 23 of the elongated body 24 and the hollow portion 28 of the linear portion 25 communicate with each other through the dilated space 52 defined by the balloon 54 of the dilation portion 50, the dilation of the dilation portion 50 is performed by the material for nerve regeneration supplied to the dilated space 52 of the balloon 54, and if the distal end 26 of the linear portion 25 penetrates the blood vessel wall due to the dilation, then, the material for nerve regeneration in the dilated space 52 is supplied to the outer wall of the blood vessel BV through the hollow portion 28 of the linear portion 25.

If the supply of the material for nerve regeneration to the outer wall of the blood vessel BV is completed, the balloon 54 deflates or is folded by extracting the liquid as the material for nerve regeneration from the dilated space 52 of the balloon 54. When the balloon 54 deflates or is folded, the distal end 26 of the linear portion 25 is removed from the blood vessel wall of the blood vessel BV by a deflating force of the balloon 54 or a restoring force to try to return to a folded state, thereby being returned to the inside of the blood vessel BV.

Here, a plurality of the linear portions 25 are provided along a circumferential direction D of the elongated body 24 (the same as the circumferential direction A of the blood vessel BV in a case where the elongated body 24 is located in the blood vessel BV) such that it is possible to supply the material for nerve regeneration to the outer wall of the blood vessel BV at plural places in the circumferential direction A of the blood vessel BV, as shown in FIG. 3(b). By doing so, it is possible to supply the material for nerve regeneration at the plural places in the circumferential direction A of the blood vessel BV. Therefore, it is possible to increase a possibility that the material for nerve regeneration may be supplied to the nerve NE regardless of the position in the circumferential direction A of the blood vessel BV, of the nerve NE which extends substantially parallel to the extension direction B of the blood vessel BV and in which neural activity is stopped or which is in an inactivation state.

In addition, as shown in FIG. 3(b), in this embodiment, four linear portions 25 are provided at positions with a central angle shifted by 90 degrees in the circumferential direction D of the elongated body 24. However, it is possible to appropriately change the number of linear portions or the central angle between the plural linear portions according to the situation of a vessel in which the supply section 22 is inserted, the type of procedure, or the like. For example, a configuration is also acceptable in which eight linear portions 25 are provided at positions with a central angle shifted by 45 degrees in the circumferential direction D of the elongated body 24.

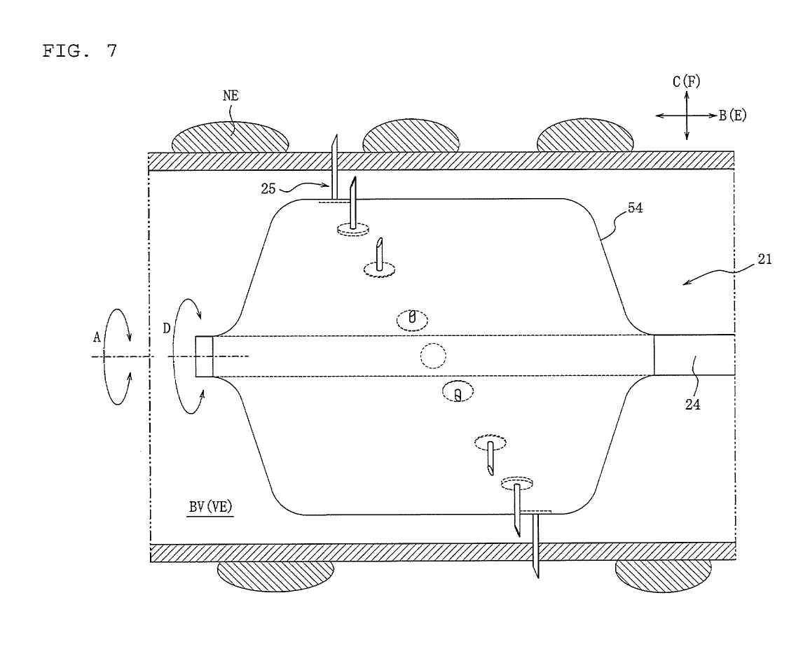

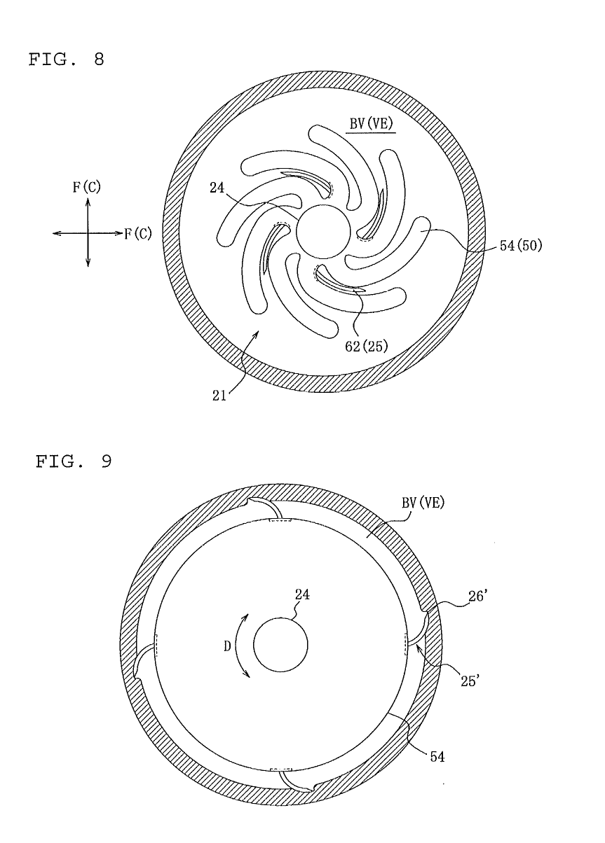

Further, a configuration is also acceptable in which the plurality of linear portions 25 are provided along an extension (i.e., longitudinal) direction E of the elongated body 24 (the same as the extension (i.e., longitudinal) direction B of the blood vessel BV in a case where the elongated body 24 is located in the blood vessel BV). With such a configuration, compared to a configuration in which the linear portion 25 is not provided in a plurality in the extension direction E of the elongated body 24, it becomes possible to more reliably supply the material for nerve regeneration to a nerve which is located in the vicinity of the outer wall of the blood vessel BV. In particular, as shown in FIG. 7, a configuration is preferable in which the positions in the circumferential direction D of the elongated body 24 of the linear portions 25 are varied according to the position in the extension direction E of the elongated body 24. In addition, in FIG. 7, the positions in the circumferential direction D of the elongated body 24 of the linear portions 25 are varied according to the position in the extension direction E of the elongated body 24 such that the linear portions 25 form a spiral shape. As described above, making the plurality of linear portions 25 puncture the blood vessel wall at a large number of positions in the circumferential direction A of the blood vessel BV is preferable in terms of performing the supply of the material for nerve regeneration. However, if too large a number of linear portions 25 puncture the blood vessel wall at only one place in the extension direction B of the blood vessel BV, the blood vessel BV causes contractile reaction at the puncture position, and thus there is a concern that local angiostenosis may occur in the blood vessel BV. Since there is a concern that such angiostenosis may impede a blood flow, it is not preferable. In this regard, if, as shown in FIG. 7, the positions of the linear portions 25 in the circumferential direction D of the elongated body 24 are varied according to the position in the extension direction E of the elongated body 24, it is possible to suppress a possibility that local angiostenosis may occur.

Here, a mounting configuration of the balloon 54 of the dilation portion 50 and the linear portion 25 will be described. The linear portion 25 in this embodiment is a needle member 62 which is provided with a hollow needle portion 60 and a flange portion 61 which is continuous to one end of the hollow needle portion 60 and extends in a direction substantially orthogonal to an extension (i.e., longitudinal) direction of the hollow needle portion 60. In the needle member 62 in this embodiment, the hollow needle portion 60 penetrates toward the outer surface from the inner surface of the balloon 54 and the flange portion 61 is solidly bonded to the inner surface of the balloon 54 by an adhesive or the like.

In addition, it is possible to form the needle member 62 with the same material as the supply section 2 (the linear portion 5) in the first embodiment described above.

Further, as a material of the balloon 54 of the dilation portion 50, it is possible to use, for example, a polyolefin (for example, polyethylene, polypropylene, polybutene, ethylene-propylene copolymer, ethylene-vinyl acetate copolymer, an ionomer, or a mixture of two kinds or more of these), a polymer material such as polyvinyl chloride, polyamide, a polyamide elastomer, polyester, a polyester elastomer, polyurethane, a polyurethane elastomer, polyimide, or a fluorine resin, or a mixture of these. Further, in addition to the above-described resin materials, as a material of the balloon 54, it is also possible to use various rubber materials such as natural rubber, butyl rubber, isoprene rubber, butadiene rubber, styrene-butadiene rubber, and silicone rubber, various thermoplastic elastomers such as polyurethane-based, polyester-based, polyamide-based, olefin-based, and styrene-based elastomers, a mixture of these, or the like.

Next, the linear portion 25 in a state where the balloon 54 of the dilation portion 50 has deflated or a state where the balloon 54 of the dilation portion 50 has been folded, and the linear portion 25 in a state where the balloon 54 of the dilation portion 50 has dilated, will be described respectively.

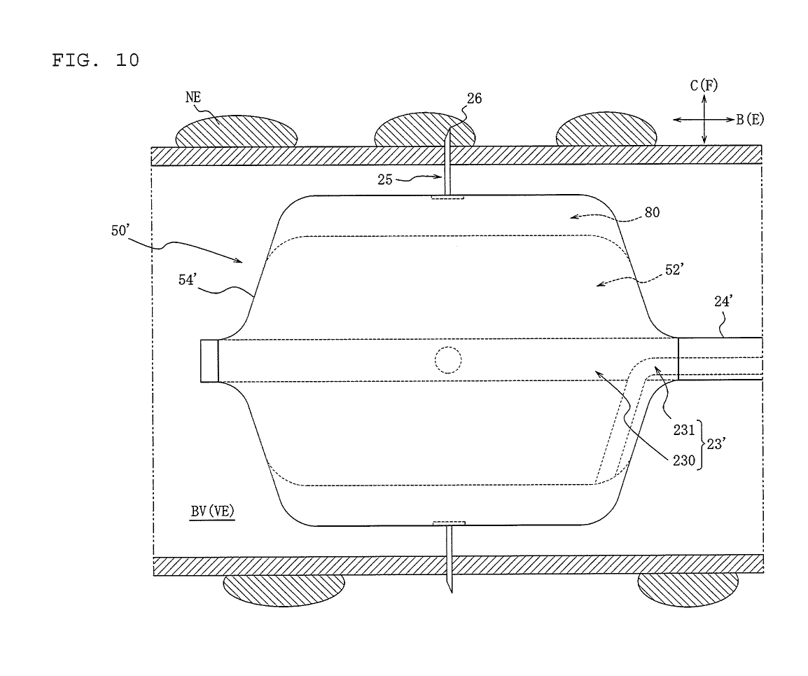

The balloon 54 in this embodiment is made of any resin material described above, and in a state before dilation, the balloon 54 is folded so as to be wound around the outer peripheral surface of the elongated body 24. The balloon 54 is inserted at a predetermined position in the blood vessel BV in the folded state. FIG. 8 is a cross-sectional view of the blood vessel BV, illustrating the balloon 54 which is in a folded state in the blood vessel BV.

The needle member 62 in this embodiment is made of a resin material, and in a state where the balloon 54 is folded, as shown in FIG. 8, the needle member 62 comprising the linear portion 25 is also elastically deformed, thereby being wound around the outer peripheral surface of the elongated body 24 in the same way as the balloon 54.

If a liquid of the material for nerve regeneration is supplied to the dilated space 52 in the balloon 54 from the above-described state where the balloon 54 is folded, the balloon 54 dilates.

The linear portion 25 elastically deformed in a state where the balloon 54 is folded changes to a posture extending in a straight line shape in a radial direction F of the elongated body 24 (the same as the radial direction C of the blood vessel BV in a case where the elongated body 24 is located in the blood vessel BV), as shown in FIGS. 3(a) and 3(b), due to a restoring force, before the balloon 54 completely dilates.

If the dilation of the balloon 54 proceeds further, the distal end 26 of the linear portion 25 comes into contact with the inner wall of the blood vessel BV, subsequently, punctures the inner wall of the blood vessel BV, and then penetrates from the inner wall to the outer wall of the blood vessel BV, whereby a state shown in FIGS. 3(a) and 3(b) is created.

In this manner, in a state where the dilation portion 50 does not dilate, the linear portion 25 extends along the circumferential direction D of the elongated body 24, and in a state where the dilation portion 50 has dilated, the linear portion 25 extends in the radial direction F of the elongated body 24, in other words, extends in a straight line shape at a predetermined angle (in this embodiment, about 90 degrees) with respect to the extension direction E of the elongated body 24.

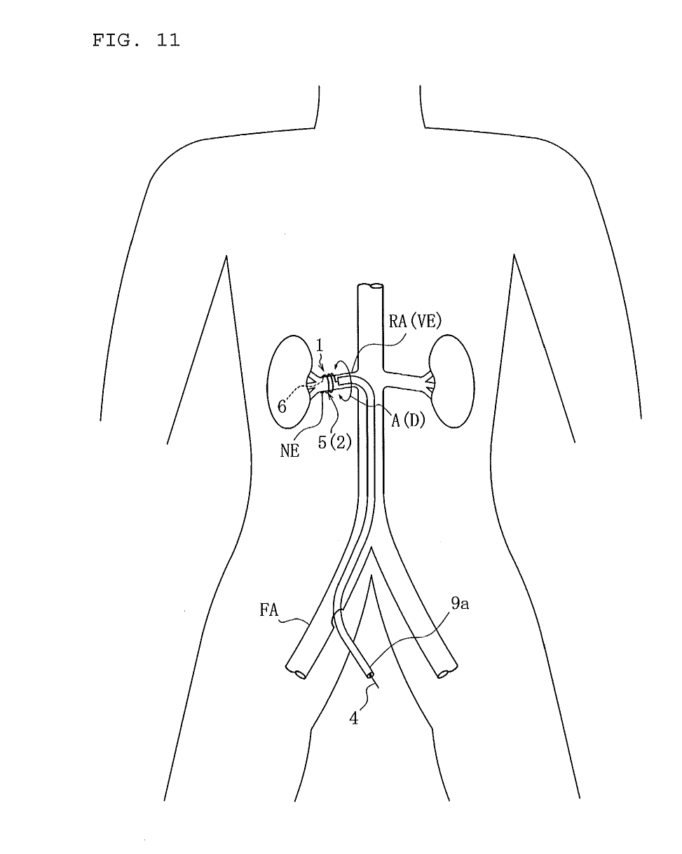

In addition, in this embodiment, the linear portion 25 is used which is elastically deformed so as to follow the outer peripheral surface of the elongated body 24 in a state where the balloon 54 has been folded, and changes to a posture extending in a straight line shape in the radial direction F of the elongated body 24 in a state where the balloon 54 has dilated. However, a configuration is also acceptable which uses a linear portion 25′ having a curved shape following the circumferential direction D of the elongated body 24 in a state where no external force acts thereon. FIG. 9 shows the linear portion 25′ in a state where the balloon 54 has dilated. As shown in FIG. 9, even in a state where the balloon 54 has dilated, the linear portion 25′ maintains a curved shape following the circumferential direction D of the elongated body 24. In a case of adopting such a configuration, by only dilating the balloon 54, there is a possibility that a distal end 26′ of the linear portion 25′ may not puncture the inner wall of the blood vessel BV. However, it is possible to make the distal end 26′ of the linear portion 25′ puncture the inner wall of the blood vessel BV by performing the axial rotation of the elongated body 24, and furthermore, it is possible to make the distal end 26′ of the linear portion 25′ penetrate from the inner wall to the outer wall of the blood vessel BV.

In addition to this, a configuration is also acceptable in which the medical material supply device 21 is provided with an outer cylindrical member which accommodates the balloon 54 which is in a deflated state or a folded state and the linear portion 25 elastically deformed so as to extend along the extension direction E of the elongated body 24 and substantially parallel to, for example, the extension direction E of the elongated body 24 inside and transports the balloon 54 and the linear portion 25 to a predetermined position in the blood vessel BV. The outer cylindrical member is a member which transports the balloon 54 and the linear portion 25 in an elastically deformed state to a predetermined position in the blood vessel BV through, for example, a guiding catheter in a state where the balloon 54 and the linear portion 25 are accommodated inside and is removed through the guiding catheter with the balloon 54 and the linear portion 25 left behind at the predetermined position. If the outer cylindrical member is removed, a posture of the linear portion 25 changes to extending in a straight line shape in the radial direction F of the elongated body 24 due to a restoring force. If a liquid is supplied to the dilated space 52 of the balloon 54 in this state, thereby dilating the balloon 54, the distal end 26 of the linear portion 25 comes into contact with the inner wall of the blood vessel BV, subsequently, punctures the inner wall of the blood vessel BV, and then penetrates from the inner wall to the outer wall of the blood vessel BV, whereby the state shown in FIGS. 3(a) and 3(b) is created.

In this manner, the configurations of the balloon 54 of the dilation portion 50 and the linear portion 25 can be realized in various ways and are not limited to the configurations shown in this embodiment.

In addition, the balloon 54 of the dilation portion 50 of this embodiment has a configuration to define only one dilated space 52. However, the balloon 54 is not limited to this configuration, and, for example, it is also possible to adopt a balloon which defines a plurality of spaces shown below.

FIG. 10 shows a dilation portion 50′ which is different in configuration from the dilation portion 50 in this embodiment. The dilation portion 50′ is provided with a balloon 54′ which defines an annular dilated space 52′ that dilates by the supply of a liquid such as a heparinized physiological salt solution thereto and an annular accommodation space 80 which is located on the outer periphery side of the dilated space 52′ and to which a liquid of a material for nerve regeneration is supplied. Here, a hollow portion 23′ of an elongated body 24′ is configured to include a first flow path 230 which supplies a liquid to the dilated space 52′, and a second flow path 231 which supplies a material for nerve regeneration to the accommodation space 80, and the first flow path 230 and the second flow path 231 are separated by a partition. With such a configuration, it is not necessary to use a large amount of liquids of a material for nerve regeneration in order to dilate the balloon 54′.

In a case of dilating the balloon 54′ of the dilation portion 50′ at a predetermined position in the blood vessel BV, first, a liquid of a material for nerve regeneration is supplied to the accommodation space 80 through the second flow path 231. Thereafter, a liquid such as a heparinized physiological salt solution is supplied to the dilated space 52′ through the first flow path 230, thereby dilating the balloon 54′ in the radial direction F of the elongated body 24′. The distal end 26 of the linear portion 25 penetrates the blood vessel wall due to the dilation. The balloon 54′ further dilates, whereby the accommodation space 80 is compressed to be sandwiched between the dilated space 52′ and the inner wall of the blood vessel BV. The material for nerve regeneration accommodated in the accommodation space 80 is extruded from the distal end 26 of the linear portion 25 by the compressive force, and as a result, the material for nerve regeneration is supplied to the outer wall of the blood vessel BV.

Next, the medical material supply device 31 as the fourth embodiment will be described with reference to FIG. 4. The medical material supply device 31 in this embodiment is different in the configuration of a linear portion from the medical material supply device 21 in the third embodiment. Here, the difference will be mainly described, and with respect to a configuration shared by the medical material supply device 21 as the third embodiment, description thereof will be omitted.

FIG. 4 is a diagram illustrating the medical material supply device 31 as the fourth embodiment. Specifically, FIG. 4(a) is a diagram illustrating the medical material supply device 31 inserted into the blood vessel BV as the vessel VE, and FIG. 4(b) is a schematic diagram illustrating a positional relationship in the circumferential direction A of the blood vessel BV between the medical material supply device 31 which is in the same state as in FIG. 4(a) and the blood vessel BV.

As shown in FIG. 4(a), the medical material supply device 31 is provided with an elongated body 34 which is inserted into the blood vessel BV from the outside of the body, and a supply section 32 is provided at an outer wall of the elongated body 34. In addition, as the elongated body 34, similar to the third embodiment, it is possible to use a tubular member such as a catheter, for example. However, in this embodiment, a guide wire having no hollow portion is used.

The supply section 32 is provided with the linear portion 35 which penetrates from the inner wall to the outer wall of the blood vessel BV, and a dilation portion 55 which retains the linear portion 35 and dilates at a predetermined position in the blood vessel BV.

The cross-section of the linear portion 35 is the same as that of the linear portion 15 in the second embodiment shown in FIG. 5. The shape of the linear portion 35 is the same shape as that of the linear portion 25 in the third embodiment. However, the linear portion 35 is different from the linear portion 25 in the third embodiment in that the linear portion 35 is a solid linear portion having no hollow portion and that the linear portion 35 is configured to include a biodegradable core 35a (the same as 15a of the second embodiment) and a layer of a material for nerve regeneration 35b (the same as 15b of the second embodiment) which includes a material for nerve regeneration provided on the outer peripheral surface of the core 35a.

In addition, the configuration of the dilation portion 55 is the same as that of the dilation portion 50 in the third embodiment described above. Specifically, the dilation portion 55 dilates at a predetermined position in the blood vessel BV, whereby a distal end 36 of the linear portion 35 penetrates from the inner wall to the outer wall of the blood vessel BV. After the distal end 36 of the linear portion 35 penetrates the blood vessel wall, the linear portion 35 is separated from the medical material supply device 31 (specifically, separated from a balloon 56 of the dilation portion 55) while remaining indwelled, and after the balloon 56 of the dilation portion 55 deflates or is folded, the medical material supply device 31 with the linear portion 35 separated therefrom is removed and recovered from the blood vessel BV.

In the linear portion 35 indwelled in the body, the layer of a material for nerve regeneration 35b in the form of a gel or the form of a solid diffuses into the body, and as a result, the material for nerve regeneration is supplied to the nerve NE which is located in the vicinity of the outer wall of the blood vessel BV, thereby promoting the regeneration of the nerve NE in which neural activity is stopped or which is in an inactivation state.

In addition, as materials of the core 35a and the layer of a material for nerve regeneration 35b, it is possible to use the same materials as the core 15a and the layer of a material for nerve regeneration 15b in the second embodiment described above. Further, as described in the second embodiment, the linear portion 35 may be configured to include the core 35a, the layer of a material for nerve regeneration 35b, and a neurotoxin layer 35c (refer to FIG. 6).

Further, with respect to the configurations of the linear portion 35, which are not particularly mentioned in this embodiment, such as a mechanism to separate the linear portion 35 from the medical material supply device 31, the disposition of the linear portion 35, and the number of linear portions 35, it is possible to adopt various configurations, as in the linear portion 15 in the second embodiment.

In addition, with respect to the configuration of the dilation portion 55, which is not particularly mentioned in this embodiment, such as the configuration of the balloon 56 of the dilation portion 55, it is possible to adopt various configurations, as in the dilation portion 50 in the third embodiment.

As described above, the medical material supply device can be realized by a variety of specific configurations and is not limited to the configurations shown in the first to fourth embodiments described above. For example, configuring a new medical material supply device by combining the configurations which are described in the descriptions of the respective embodiments belongs to the technical scope of the present disclosure.

Next, a method of supplying a material for nerve regeneration to the nerve NE which is located in the vicinity of the outer wall of the vessel VE by using the medical material supply devices 1, 11, 21, and 31 as the first to fourth embodiments described above will be described. In addition, here, description will be performed by exemplifying a renal artery RA as the vessel VE.

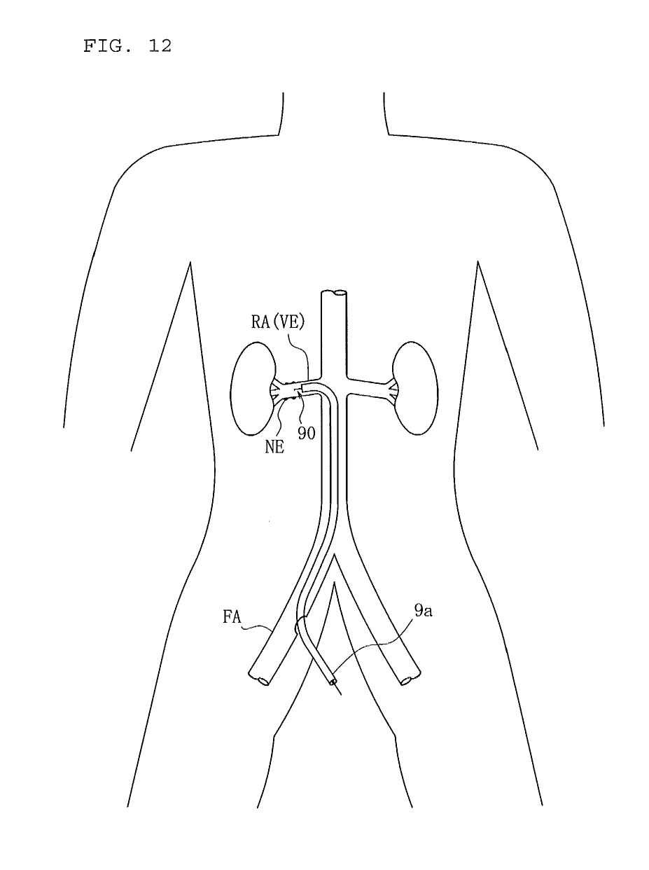

FIG. 11 is a diagram describing treatment to supply a material for nerve regeneration to a renal artery sympathetic nerve NE which is in an inactivation state, by using the medical material supply device 1 as the first embodiment. This treatment includes a step of inserting the medical material supply device 1 into the renal artery RA, thereby introducing the medical device 1 at a predetermined position in the renal artery RA, and a step of making the distal end 6 of the linear portion 5 provided in the medical material supply device 1 penetrate from the inner wall to the outer wall of the renal artery RA at the predetermined position in the renal artery RA, thereby supplying a material for nerve regeneration to an outer wall of the renal artery RA from the distal end 6 of the linear portion 5.

Specifically, an operator inserts the guiding catheter 9a into a femoral artery FA of a patient in advance and makes the distal end 10 of the guiding catheter 9a arrive at the renal artery RA. A guide wire (not shown) is used to make the guiding catheter 9a arrive at the renal artery RA. The guiding catheter 9a is tubular and can insert the medical material supply device 1 therein.

First, the operator inserts the medical material supply device 1 into the guiding catheter 9a and sends the medical material supply device 1 to the renal artery RA. That is, the medical material supply device 1 is transported to a predetermined position in the renal artery RA beyond the distal end 10 of the guiding catheter 9a and is deformed in a spiral shape by a restoring force at the predetermined position, thereby coming into contact with an inner wall of the renal artery RA. In this state, the operator performs the axial rotation of the elongated body 4 of the medical material supply device 1, thereby making the distal end of the elongated body 4 (the distal end 6 of the linear portion 5) penetrate from the inner wall to the outer wall of the renal artery RA. The operator further performs the axial rotation of the elongated body 4 in the same direction, whereby the distal end 6 of the linear portion 5 having a spiral shape advances to the kidney side, thereby making the linear portion 5 be wound around the outer wall of the renal artery RA, whereby the state shown in FIG. 2 can be created.

Subsequently, the operator sends a material for nerve regeneration through the inside of the elongated body 4 from the proximal end of the elongated body 4, which is located outside the body, and retreats the distal end 6 of the linear portion 5 by performing the axial rotation of the elongated body 4 in the opposite direction to the direction in a case of advancing the distal end 6 of the linear portion 5 to the kidney side, while supplying the material for nerve regeneration in the form of a gel or the form of a liquid from the distal end 6 of the linear portion 5 to the outer wall of the renal artery RA. In this way, it is possible to supply the material for nerve regeneration to the outer wall of the renal artery RA over the entire area in a circumferential direction A of the renal artery RA, and therefore, it becomes possible to more reliably supply the material for nerve regeneration to the nerve NE which is in an inactivation state.

Thereafter, the treatment to supply the material for nerve regeneration is completed by returning the distal end 6 of the linear portion 5 into the renal artery RA and removing the medical material supply device 1 to the outside of the body through the guiding catheter 9a.

In addition, as described in the description of the first embodiment, as the transport member 9, an outer cylindrical member may be used.

Further, even in the case of treatment to supply the material for nerve regeneration to the renal artery sympathetic nerve NE which is in an inactivation state by using the medical material supply device 11 as the second embodiment, steps up to and including the step of winding the linear portion 15 around the outer wall of the renal artery RA are the same as in the above-described treatment using the medical material supply device 1 as the first embodiment. In the case of the treatment using the medical material supply device 11 as the second embodiment, thereafter, the indwelled linear portion 15 which has been separated from the medical material supply device 11, and the medical material supply device 11 with the linear portion 15 separated therefrom, are removed to the outside of the body through the inside of the guiding catheter 9a, whereby the treatment is completed.

In the case of the treatment to supply the material for nerve regeneration to the renal artery sympathetic nerve NE which is in an inactivation state by using the medical material supply device 21 as the third embodiment, the supply section 22 is sent to a predetermined position in the renal artery RA through the guiding catheter and the balloon 54 of the dilation portion 50 dilates at the predetermined position. The balloon 54 of the dilation portion 50 dilates, whereby the distal end 26 of the linear portion 25 penetrates from the inner wall to the outer wall of the renal artery RA. In this state, the operator sends a liquid of the material for nerve regeneration into the elongated body 24, thereby supplying the material for nerve regeneration from the distal end 26 of the linear portion 25 to the outer wall of the renal artery RA. Then, if the supply of the material for nerve regeneration is completed, the balloon 54 of the dilation portion 50 deflates or is folded and the medical material supply device 21 is recovered through the guiding catheter, whereby the treatment is completed.

Even in the case of treatment to supply the material for nerve regeneration to the renal artery sympathetic nerve NE which is in an inactivation state by using the medical material supply device 31 as the fourth embodiment, steps up to and including the step of making the distal end 36 of the linear portion 35 penetrate from the inner wall to the outer wall of the renal artery RA are the same as in a case of using the medical material supply device 21 as the third embodiment described above. In the case of the treatment using the medical material supply device 31 as the fourth embodiment, thereafter, the linear portion 35 which has been separated from the medical material supply device 31, and the medical material supply device 31 with the linear portion 35 separated therefrom, are removed to the outside of the body through the inside of the guiding catheter, whereby the treatment is completed.

Finally, treatment to regenerate the nerve NE which is in an inactivation state due to denervation treatment after denervation is performed will be described. This is because, as described in the description of the second embodiment, by regenerating the renal artery sympathetic nerve NE which is in an inactivation state due to the denervation after the denervation of the renal artery sympathetic nerve NE is performed, the possibility for the effect of a fall in blood pressure being obtained is high.

FIG. 12 is a diagram illustrating treatment to perform the denervation of the renal artery sympathetic nerve NE. After the treatment to perform the denervation shown in FIG. 12, the above-described treatment to supply the material for nerve regeneration to the renal artery sympathetic nerve NE which is in an inactivation state, which uses the medical material supply devices 1, 11, 21, and 31 as the first to fourth embodiments, is performed (refer to FIG. 11). That is, this treatment includes a step of stopping the neural activity of the renal artery sympathetic nerve NE which extends along the outer wall of the renal artery RA, a step of inserting the medical material supply device 1 into the renal artery RA, thereby introducing the medical material supply device 1 at a predetermined position in the renal artery RA, and a step of making the distal end 6 of the linear portion 5 provided in the medical material supply device 1 penetrate from the inner wall to the outer wall of the renal artery RA at the predetermined position in the renal artery RA, thereby supplying the material for nerve regeneration from the distal end 6 of the linear portion 5 to the outer wall of the renal artery RA.

As shown in FIG. 12, the step of stopping the neural activity of the renal artery sympathetic nerve NE which extends along the outer wall of the renal artery RA can be performed by using a cauterization device 90 which stops the neural activity by cauterizing the renal artery sympathetic nerve NE, for example. An operator inserts a distal end of the cauterization device 90 at a predetermined position in the renal artery RA through the guiding catheter 9a. In this state, the operator irradiates the renal artery sympathetic nerve NE to be cauterized, with energy (for example, ultrasonic waves) for cauterization by using the cauterization device 90, thereby performing denervation. If the denervation by the cauterization device 90 is completed, the operator removes the cauterization device 90 to the outside of the body through the guiding catheter 9a.

Subsequently, the operator inserts any one of the medical material supply devices 1, 11, 21, and 31 as the first to fourth embodiments into the renal artery RA through the guiding catheter 9a and supplies the material for nerve regeneration to the outer wall of the renal artery RA, as described above (refer to FIG. 11).

In addition, here, the denervation treatment of the renal artery sympathetic nerve NE is performed by the cauterization device 90 which is different from the medical material supply devices 1, 11, 21, and 31 as the first to fourth embodiments. However, it is also possible to perform the denervation of the renal artery sympathetic nerve NE by using the medical material supply devices 1, 11, 21, and 31 as the first to fourth embodiments.

For example, if neurotoxin is supplied from the linear portion 5 of the medical material supply device 1 as the first embodiment to the renal artery sympathetic nerve NE and a material for nerve regeneration is then supplied from the linear portion 5 to the renal artery sympathetic nerve NE, both the denervation and the supply of the material for nerve regeneration can be performed by using the medical material supply device 1. Further, the hollow portion 3 of the elongated body 4 in the medical material supply device 1 may be configured with two flow paths, a flow path for neurotoxin and a flow path for a material for nerve regeneration, and the supply of the neurotoxin and the supply of the material for nerve regeneration are performed at the same time by supplying both the neurotoxin and the material for nerve regeneration from the separate flow paths to the renal artery sympathetic nerve NE. In addition, even in the medical material supply device 21 as the third embodiment, it is possible to use the same method.

In addition to this, as described in the description of the second embodiment or the description of the fourth embodiment, the linear portions 15 and 35 in the medical material supply devices 11 and 31 as the second embodiment and the fourth embodiment are configured to include the biodegradable cores 15a and 35a, the layers of a material for nerve regeneration 15b and 35b, and the neurotoxin layers 15c and 35c, whereby it is possible to perform the supply of both the neurotoxin and the material for nerve regeneration by using the medical material supply device 11 or 31.

The detailed description above describes a medical material supply device. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical material supply device that is inserted into a vessel, comprising:
a supply section configured to penetrate from an inner wall to an outer wall of the vessel at a predetermined position in the vessel, thereby being able to supply a material for nerve regeneration to the outer wall of the vessel; and
an elongated body which is inserted into the vessel,
wherein the supply section is provided with a linear portion which is provided at the elongated body and is configured to penetrate from the inner wall to the outer wall of the vessel.

2. The medical material supply device according to claim 1,
wherein the linear portion is provided with a biodegradable core and a layer of a material for nerve regeneration which includes the material for nerve regeneration provided on an outer peripheral surface of the core.

3. The medical material supply device according to claim 2,
wherein the linear portion is configured to be separated from the medical material supply device while remaining indwelled after penetrating from the inner wall to the outer wall of the vessel at the predetermined position.

4. The medical material supply device according to claim 1,
wherein the elongated body defines a hollow portion inside thereof, and
the linear portion defines a hollow portion which communicates with the hollow portion of the elongated body and also communicates with a distal end of the linear portion, and thus the material for nerve regeneration is supplied from the hollow portion of the elongated body to the outer wall of the vessel through the hollow portion of the linear portion.

5. The medical material supply device according to claim 1,
wherein the linear portion comprises a distal end portion which includes a distal end of the elongated body, and has a spiral shape.

6. The medical device according to claim 1,
wherein the linear portion extends at a predetermined angle with respect to an extension direction of the elongated body.

7. The medical material supply device according to claim 6,
wherein the supply section is provided with a dilation portion which retains the linear portion and dilates at the predetermined position in the vessel, and
wherein the distal end of the linear portion is configured to penetrate from the inner wall to the outer wall of the vessel when the dilation portion dilates.

8. The medical material supply device according to claim 7,
wherein the elongated body defines a hollow portion inside thereof, and
the dilation portion is provided with a balloon which is provided at an outer wall of the elongated body and defines a dilated space to which a liquid is supplied from the hollow portion through a hole connecting the outer wall and an inner wall of the elongated body.

9. The medical material supply device according to claim 2,
wherein the linear portion comprises a distal end portion which includes a distal end of the elongated body, and has a spiral shape.

10. The medical material supply device according to claim 3,
wherein the linear portion comprises a distal end portion which includes a distal end of the elongated body, and has a spiral shape.

11. The medical material supply device according to claim 4,
wherein the linear portion comprises a distal end portion which includes a distal end of the elongated body, and has a spiral shape.

12. The medical device according to claim 2,
wherein the linear portion extends at a predetermined angle with respect to an extension direction of the elongated body.

13. The medical device according to claim 3,
wherein the linear portion extends at a predetermined angle with respect to an extension direction of the elongated body.

14. The medical device according to claim 4,
wherein the linear portion extends at a predetermined angle with respect to an extension direction of the elongated body.

15. A medical treatment method using a medical material supply device, the method comprising:
inserting the medical material supply device into a vessel;
penetrating from an inner wall to an outer wall of the vessel with a linear portion of the medical material supply device; and
supplying medical material to the vessel with the linear portion.

16. The medical treatment method according to claim 15, further comprising, after penetrating from the inner wall to the outer wall of the vessel with the linear portion of the medical material supply device, winding the linear portion around the vessel.

17. The medical treatment method according to claim 15, further comprising, after penetrating from the inner wall to the outer wall of the vessel with the linear portion of the medical material supply device, separating the linear portion from the medical material supply device.

18. The medical treatment method according to claim 15, further comprising penetrating from the inner wall to the outer wall of the vessel with a plurality of linear portions.

19. The medical treatment method according to claim 15, wherein the linear portion has a hollow portion and the medical material is supplied to the vessel by being supplied to the hollow portion of the linear portion.

20. The medical treatment method according to claim 15, wherein the linear portion is provided with an outer layer of the medical material which is supplied to the vessel.
